(19) United States
(12) Patent Application Publication
(10) Pub. No.: US 2025/0205314 A1
(43) Pub. Date: Jun. 26, 2025

(54) CGAS SUPER-ENZYMES FOR CANCER IMMUNOTHERAPY

(71) Applicant: ECOLE POLYTECHNIQUE FEDERALE DE LAUSANNE (EPFL), Lausanne (CH)

(72) Inventors: Andrea ABLASSER, Lausanne (CH); Natasha SAMSON, Lausanne (CH); Alexander KELLER, Lausanne (CH)

(21) Appl. No.: 18/840,440

(22) PCT Filed: Feb. 20, 2023
(86) PCT No.: PCT/EP2023/054168

§ 371 Date: Aug. 21, 2024

(30) Foreign Application Priority Data
Feb. 22, 2022 (EP) 22158014.5

(51) Int. Cl. A61K 38/45; A61K 35/17; A61P 35/00

(52) U.S. Cl. CPC A61K 38/45; A61K 35/17; A61P 35/00; C12Y 207/07

(57) ABSTRACT

The present invention relates to anti-tumor lymphocytes, and a variant cyclic GMP-AMP synthase (cGAS) carrying an amino acid substitution of one or both arginines at amino acid positions 255 and 236 and/or an amino acid substitution of one or both lysines at amino acid positions 254 and 258; or a nucleic acid molecule encoding said variant cGAS for use in the treatment of a tumor in a subject.

The present invention relates to anti-tumor lymphocytes, and a variant cyclic GMP-AMP synthase (cGAS) carrying an amino acid substitution of one or both arginines at amino acid positions 255 and 236 and/or an amino acid substitution of one or both lysines at amino acid positions 254 and 258; or a nucleic acid molecule encoding said variant cGAS for use in the treatment of a tumor in a subject.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

New therapies that promote anti-tumour immunity have recently been developed. Most of these immunomodulatory approaches have focused on enhancing T-cell responses, either by targeting inhibitory pathways with immune checkpoint inhibitors, or by targeting activating pathways, as with chimeric antigen receptor T cells or bispecific antibodies (Demaria et al. (2019), Nature, 574; 54-56).

For instance, early structural and functional studies on cyclic guanosine monophosphate-adenosine monophosphate (GMP-AMP) synthase (cGAS) revealed that this enzyme interacts with dsDNA in a sequence-independent manner. cGAS initiates innate immune responses following microbial infection, cellular stress and cancer. Upon activation by double-stranded DNA, cytosolic cGAS produces 2′3′ CGMP-AMP, which triggers the induction of inflammatory cytokines and type I interferons. cGAS is also present inside the cell nucleus, which is replete with genomic DNA, where chromatin has been implicated in restricting its enzymatic activity. The broad biological roles of intracellular DNA sensing create new opportunities for the exploration and therapeutic manipulation for the prevention and treatment of multiple human diseases. Initial successes with therapies targeting the immunostimulatory effects of the cGAS-STING pathway suggest a major clinical impact in areas of cancer immunotherapy and vaccine development (Pathare et al. (2020), Nature; 587 (7835):668-672 and Ablasser and Chen (2019), Science, 363 (6431):eaat8657). Multiple STING agonists were developed for cancer therapy study with highly promising results achieved in pre-clinical work. Recent progress in the mechanistic understanding of STING pathway in IFN production and T cell priming indicates its promising role for cancer immunotherapy (Jiang et al. (2020), Journal of Hematology & Oncology, 13: 81).

Also adoptive T cell therapy, or the infusion of disease-targeting T cells as the therapeutic agent, has demonstrated remarkable potential to treat advanced-stage cancers. In this novel treatment paradigm, primary human T cells are genetically modified to express tumour-specific receptors. The engineered T cells typically express either a chimeric antigen receptor (CAR) or T cell receptor (TCR) and mount a tumour-specific immune response when infused into the patient. CAR-T cells targeting the CD19 antigen expressed in B cells became the first gene-therapy product to be approved by the FDA. Patients with relapsed or refractory B cell malignancies achieved complete remission rates of up to 90% (Hou et al. (2020), Nature Reviews|Drug Discovery; 20:531-550).

Despite the above-discussed success of therapies promoting anti-tumour immunity, there is an ongoing need for further anti-tumor treatment options. This need is addressed by the present invention.

The present invention therefore relates to anti-tumor lymphocytes and a variant cyclic GMP-AMP synthase (cGAS) carrying an amino acid substitution of one or both arginines at amino acid positions 255 and 236 and/or an amino acid substitution of one or both lysines at amino acid positions 254 and 258; or a nucleic acid molecule encoding said variant cGAS for use in the treatment of a tumor in a subject.

In this context an amino acid substitution of one or both arginines at amino acid positions 255 and 236 is preferred over an amino acid substitution of one or both lysines at amino acid positions 254 and 258.

A lymphocyte is a type of white blood cell in the immune system of jawed vertebrates. Lymphocytes include, innate lymphoid cells (ILCs, i.e. innate counterparts of T cells that contribute to immune responses by secreting effector cytokines and regulating the functions of other innate and adaptive immune cells), natural killer cells (which function in cell-mediated, cytotoxic innate immunity), T cells (for cell-mediated, cytotoxic adaptive immunity), and B cells (for humoral, antibody-driven adaptive immunity).

An anti-tumor lymphocyte (or an anti-tumor effector lymphocyte) is a lymphocyte capable of eliciting a cytolytic response that can cause tumor cell death. These lymphocytes are specializing in and equipped for tumor cell elimination. The first category encompasses clonally expanded T lymphocytes expressing a unique T cell receptor (TCR) and recognizing tumor epitopes in the context of the major histocompatibility complex (MHC) molecules. These T cells, optionally together with B cells producing tumor-specific antibodies and dendritic cells (DC) processing and presenting tumor epitopes, can mediate an adaptive immunity against tumors. The second category of effector cells includes natural killer (NK) cells, NK-T cells, and macrophages (M). These cells are not restricted by the MHC molecules in their interactions with tumor targets, and they mediate innate immunity. Each type of effector cells, whether specific or nonspecific, contains subsets of cells at different stages of differentiation and activation. This means that each type of effector potentially able to target tumor cells contains a heterogeneous mix of cells with distinct functional capabilities, depending on their stage of differentiation, maturation, and/or activation (Holland, Frei; Cancer Medicine; 6th edition. chapter “Antitumor Effector Cells in Humans”). All these types of anti-tumor lymphocytes are applicable in accordance with the present invention.

Cyclic GMP-AMP synthase (cGAS, cGAMP synthase) belongs to the nucleotidyltransferase family. This enzyme is a cytosolic DNA sensor that activates a type-I interferon response. It is part of the cGAS-STING DNA sensing pathway. It binds to microbial DNA as well as self-DNA that invades the cytoplasm, and catalyzes cGAMP synthesis. cGAMP then functions as a second messenger that binds to and activates the endoplasmic reticulum protein STING to trigger type-I IFNs production.

Human variant cyclic GMP-AMP synthase (cGAS) carrying an amino acid substitution of one or both arginines at amino acid positions 255 and 236 are untethered and constitutively active. For instance, Human variant R236E cGAS and human variant R255E mutants were reported to be untethered and constitutively active, with R255E hcGAS constitutively producing over 100 times more cGAMP than wild-type human GAS (Volkman et al (2019), eLife; 8: e47491). In addition, R236A or R255A mutations of human cGAS are known to impair the binding between human cGAS and the nucleosome which largely relieves the nucleosome-mediated inhibition of human cGAS activity (Cao et al. (2020), Cell Research, 30:1088-1097). By contrast, wild-type human cGAS is bound to nucleosome within the cell nucleus which inhibits the activation of cGAS through blocking the interaction of cGAS with ligand dsDNA and also disrupting cGAS dimerization.

It is believed that an amino acid substitution of one or both lysines at amino acid positions 254 and 258 results in constitutively active variant cGAS just as an amino acid substitution of one or both arginines at amino acid positions 255 and 236. This likewise applies to an amino acid substitution of one or both lysines at amino acid positions 254 and 258 in addition to an amino acid substitution of one or both arginines at amino acid positions 255 and 236. This is because the acidic patch binding by residues K254 and K258 is believed to be responsible for stabilizing the complex of cGAS with the nucleosome. The cGAS variants K254A and K258A both show partly impaired binding with the nucleosome (Cao et al. (2020), Cell Research; 30:1088-1097).

For the above reasons, a variant cGAS carrying an amino acid substitution of one or both arginines at amino acid positions 255 and 236 and/or an amino acid substitution of one or both lysines at amino acid positions 254 and 258 of the invention designates a constitutively active variant cGAS. In this respect “constitutively active” means that the variant cGAS is capable of producing cGAMP when present within a cell or subject. As will be further discussed herein below, a variant cGAS may not comprise the intrinsically disordered region (IDR) region that can be found at the N-terminus of cGAS since this region is not necessary for the activity of cGAS, in particular for catalyzing cGAMP synthesis.

The term “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) which is another embodiment of the nucleic acid molecule means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrids molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analogue, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8:1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule of the invention may additionally comprise promoters, enhancers, response elements, signal sequences, polyadenylation sequences, introns, 5′- and 3′-non-coding regions, and the like.

The nucleic acid molecule according to the invention encodes a variant cyclic GMP-AMP synthase (cGAS) carrying an amino acid substitution of one or both arginines at amino acid positions 255 and 236 an/or an amino acid substitution of one or both lysines at amino acid positions 254 and 258. As will be further detailed herein below it is preferred that the nucleic acid molecule of the invention is genomic DNA or mRNA, wherein the genomic DNA might have been added into the genome of a cell, preferably an anti-tumor lymphocyte. In the case of mRNA, the nucleic acid molecule may in addition comprise a poly-A tail.

The nucleic acid molecule according to the invention preferably encodes a variant cyclic GMP-AMP synthase (cGAS) carrying an amino acid substitution of one or both arginines at amino acid positions 255 and 236 and/or an amino acid substitution of one or both lysines at amino acid positions 254 and 258 in expressible form. This means that nucleic acid molecule encodes the variant cGAS in a form that can be transcribed and translated into the variant cGAS enzyme with a cell or subject. A non-limiting example of such a nucleic acid molecule is an expression vector, as will be further explained herein below.

A tumor is an abnormal benign or malignant new growth of tissue that possesses no physiological function and arises from uncontrolled usually rapid cellular proliferation.

The subject herein is preferably a mammal and more preferably a primate.

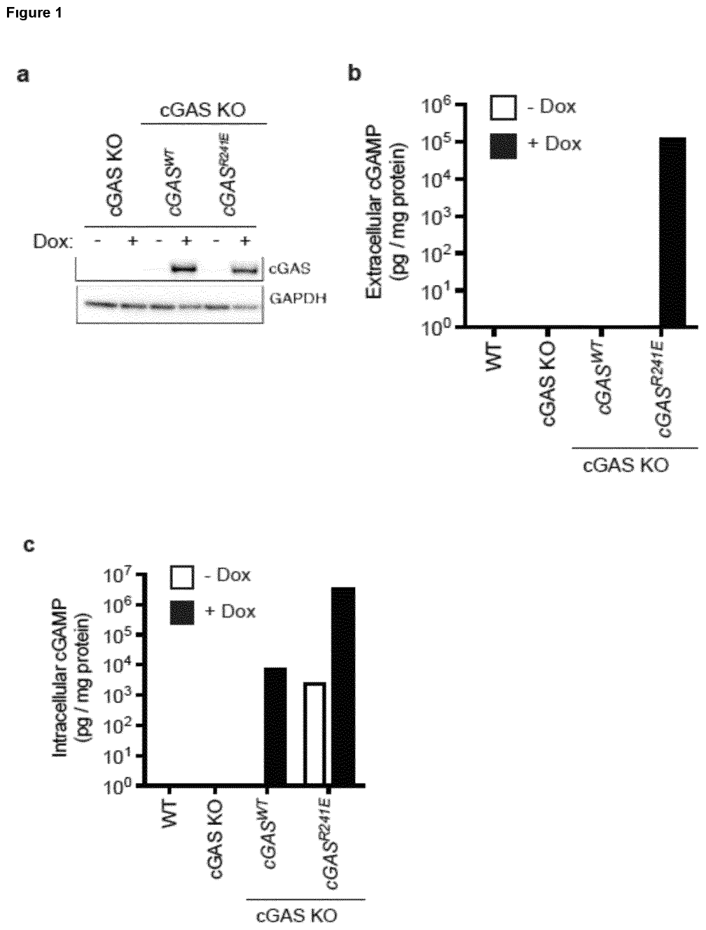

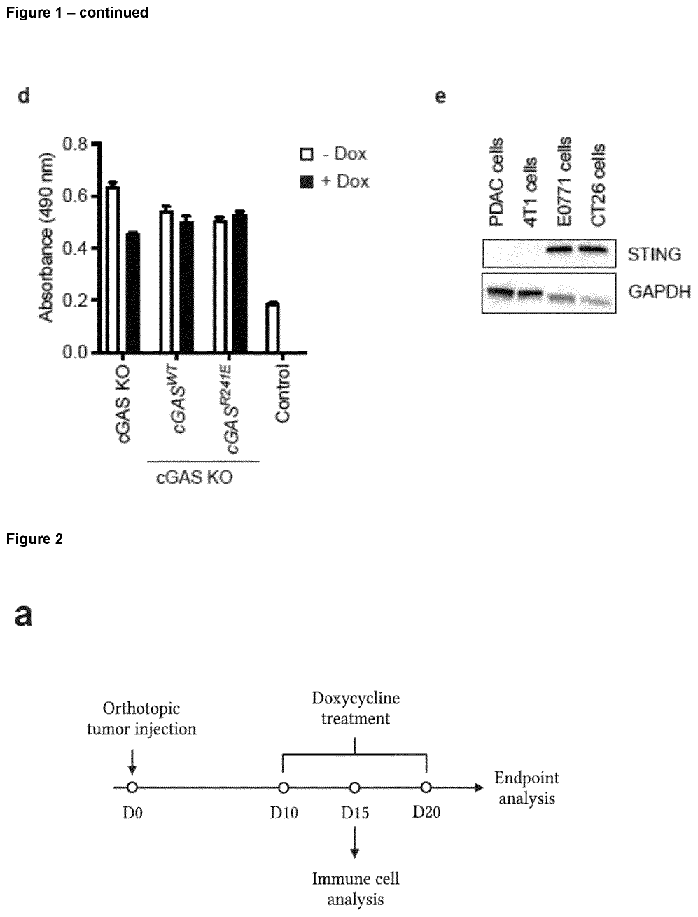

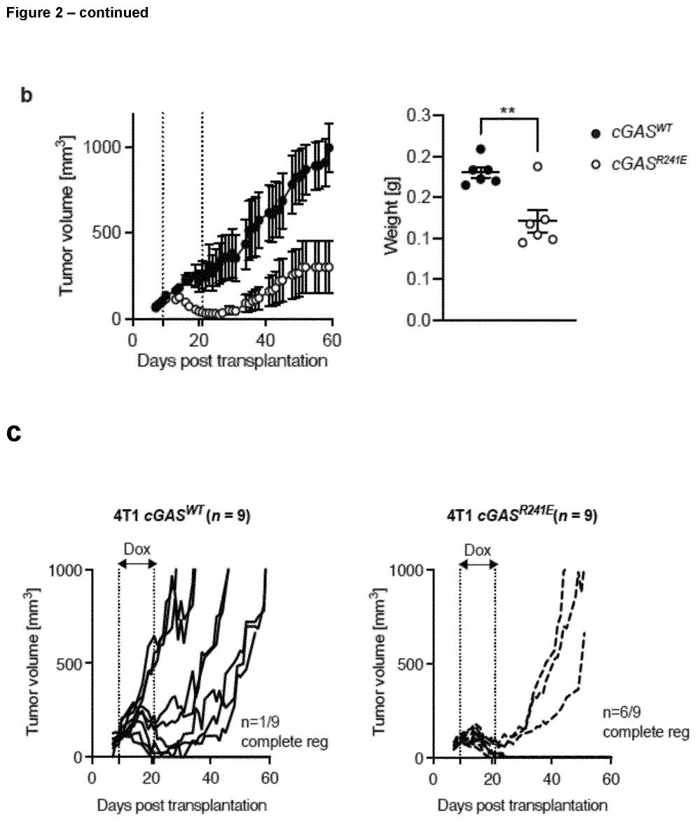

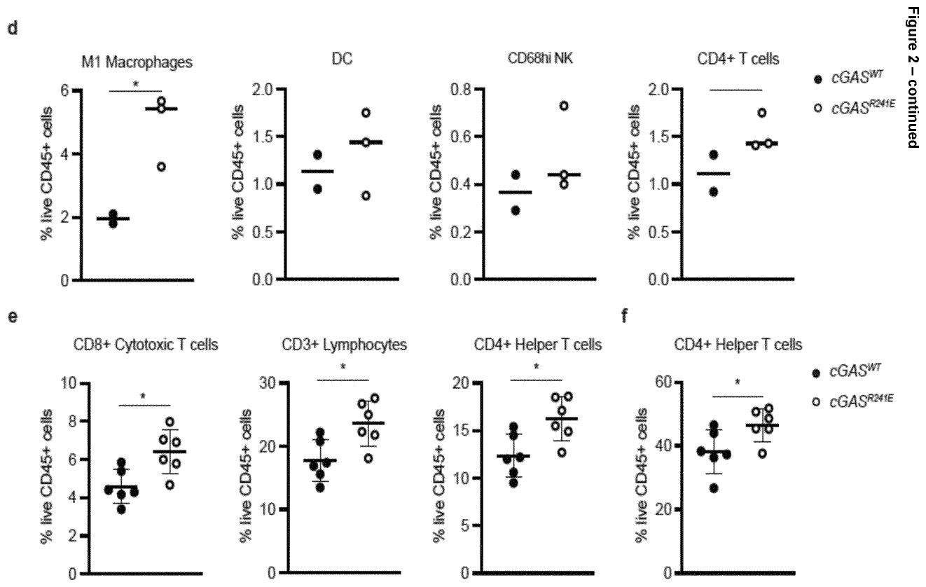

A variant mouse cGAS carrying the amino acid substitution R241E is the species ortholog of the human cGAS carrying the amino acid substitution R255E. It is demonstrated in Example 1 and FIG. 1 that mouse cGAS R241E when introduced into mouse tumor cells is active. This is evidenced by the production of cGAMP. Example 2 and FIG. 2 furthermore show that mouse cGAS R241E leads to tumor regression as well as the activation of immune cells. In particular the high number of CD4+and CD8+provides evidence that the combined use of anti-tumor lymphocytes, such as CAR T cells and an active cGAS variant is beneficial. It is expected that the active cGAS variant not only reduces the tumor load but also enhances and promotes the anti-tumor activity of the anti-tumor lymphocytes by activating them. The combined use of an active cGAS variant and anti-tumor lymphocytes, and in particular of anti-tumor lymphocytes expressing an active cGAS variant is therefore expected to provide a therapeutic effect that exceeds the cumulative effects of the active cGAS variant and the anti-tumor lymphocytes alone. This will be explained in more detail herein below on the basis of CAR-T cells expressing the active cGAS variant that will be called “cGAMP CAR-T cells”.

Owing to the potent immunostimulatory capacity of cGAMP, cGAMP CAR-T cells can activate a vigorous endogenous anti-tumor immune response. Further, it is expected that cGAMP CAR-T cells show improved cell-intrinsic functions. Together, equipped with these functional properties cGAMP CAR-T cells address major current barriers in directing CAR-T cells against solid tumor STING agonists and have excited a lot of interest in augmenting anti-tumor immune responses. Preclinical models have reported that if combined with a STING agonist treatment, CAR-T cells show improved potency against solid tumors. In addition, ex vivo pre-treatment with a STING agonist can promote T cell stemness and enhance cell intrinsic functions of CAR-T cells. However, compared to combining STING agonists with conventional CAR-T cells, the functionalization of CAR-T cells with cGAS super-enzymes bears multiple and significant advantages. Rather than simple achieving the activation of STING in the tumor microenvironment (TME), cGAS super-enzyme will engender T cells themselves with emergent stimulatory properties that will result in marked improved therapeutic potency.

First-generation STING agonists, i.e. modified versions of natural 2′-3′cGAMP, are undergoing clinical testing with mixed overall results with only a minority of patients responding to the treatment. Owing to their metabolic instability, these agonists must be administered intratumorally and, thus are limited to certain types of solid tumors. Moreover, intratumor administration in itself bears considerable disadvantages, such as the sub-optimal intratumoral permeation/diffusion of the agonists with considerable variation in drug availability and the impossibility to reach (small) metastatic lesions in advanced stage cancer patients.

To overcome these limitations of intratumoral injection, improved formulations of first STING agonists are being developed as are second generation STING agonists, which are based on entirely distinct chemical matter. However, an important remaining challenge of systemically applied STING agonists is the achievement of high intratumoral delivery while at the same time minimizing systemic, toxic side effects. cGAMP CAR-T cells are expected to overcome this critical barrier. First, cGAMP CAR-T cells will preferential deliver cGAMP in the tumor because of intratumoral enrichment of the CAR-T cells. Second, the low metabolic stability of natural 2′-3′cGAMP (i.e., endogenous cGAS product) secreted by cGAMP CAR-T cells will reduce the risk of systemic dissipation.

Another important consideration for the application of synthetic STING agonists is the finding of an optimized dose. In fact, STING agonists have been shown to exhibit bell-shaped efficacy curves after intratumoral administration (12). One reason for the reduced potency of high local doses of synthetic STING agonists is thought to be their ability to trigger robust T cell death. Importantly, extracellular natural cGAMP is tuned to act more “modestly” and, lacking pro-apoptotic properties cGAMP CAR-T cells are not expected to cause significant T cell death responses in the tumor. Overall, compared to STING agonists, cGAMP CAR-T cells are expected to achieve superior pharmacodynamical and-kinetically properties: The continuous secretion of natural 2′-3′cGAMP will enable a steady, local optimal concentration of the immune-stimulator ligand across the tumor bed.

In particular, the coupling of the delivery of cGAMP with the killing of antigen-expressing tumor cells in one “cell therapy”, bears significant advantages over separate delivery regimens.

First, engagement of the cGAS-STING pathway in cancer cells can lead to immunosuppression and the promotion of metastasis representing a significant disadvantage of STING agonists for the treatment of solid tumors. The co-delivery of cGAMP packaged within CAR-T cells, by contrast, would circumvent this antagonistic effect. Through the killing of antigen-matched tumors, CAR-T cells would reduce the availability of viable cancer cells that could mount cGAMP-dependent immune-regulatory responses. Instead, the cGAMP response would be more focused on the non-cancer cell compartment in the TME, thus allowing to mitigate the immunosuppressive effect of cGAMP. Second, CAR-T cell delivery will also ensure the proper timing of stimulation of antigen-presenting cells in the tumor with cancer cell death. The coordination of immune-stimulation of DCs with the release of tumor antigen is expected to be beneficial for an improved priming of endogenous T cells response against non-CAR antigens.

In accordance with a preferred embodiment of the invention the anti-tumor lymphocytes are T-cells or NK cells.

A T-cell or T-lymphocyte can be distinguished from other lymphocytes by the presence of a T-cell receptor (TCR) on their cell surface. One of the functions of T-cells is mediating immune-mediated cell death, and it is carried out by two major subtypes: CD8+ “killer” and CD4+ “helper” T-cells. CD8+ T cells are cytotoxic which means that they are able to directly kill selected cell. These selected cells are in accordance with the invention tumor cells, virus-infected cells, as well as cancer cells. CD4+ cells function as “helper cells”. Unlike CD8+ killer T-cells, these CD4+ helper T-cells function by further activating memory B cells and cytotoxic T-cells, which leads to a larger immune response which is in accordance with the invention directed against tumor cells. The specific adaptive immune response regulated by the T-helper cell depends on its subtype, which is distinguished by the types of cytokines they secrete. The T-cells are preferably a CD8+ killer T-cells or mixture of CD8+ killer and CD4+ helper T-cells.

A natural killer (NK) cell is a type of cytotoxic lymphocyte being critical to the innate immune system that belong to the rapidly expanding family of innate lymphoid cells (ILC) and represent 5-20% of all circulating lymphocytes in humans. The role of NK cells in innate immune system is analogous to that of cytotoxic T-cells in the vertebrate adaptive immune response. NK cells provide rapid responses to virus-infected cells and other intracellular pathogens acting at around 3 days after infection and respond to tumor formation. Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. They were named “natural killers” because they do not require activation to kill cells that are missing “self” markers of MHC class 1. This role is especially important because harmful cells that are missing MHC I markers cannot be detected and destroyed by other immune cells, such as T-cells.

In accordance with another preferred embodiment of the invention the anti-tumor lymphocytes are chimeric antigen receptor T-cells (CAR T-cells), T-cell-receptor-engineered T-cells (TCR T-cells), chimeric antigen receptor NK-cells (CAR NK-cells), NK cell receptor-engineered NK cells (NCR NK-cells), TCR/CAR hybrid T-cells, NCR/CAR hybrid NK-cells or tumor-infiltrating lymphocytes (TILs).

Chimeric antigen receptor T-cells (CAR) T-cells are T-cells that have been genetically engineered to produce a chimeric T cell receptor (CAR) for use in immunotherapy. The receptors are chimeric because they combine both antigen-binding and T-cell activating functions into a single receptor. CAR-T cell therapy uses T-cells engineered with CARs for cancer therapy. The premise of CAR-T immunotherapy is to modify T-cells to recognize tumor cells in order to more effectively target and destroy them. in order to generate CAR T-cells. T-cells are harvested from subject, genetically altered, and then infused into patients to attack a tumor in a subject. CAR T-cells can be both CD4+ and/or CD8+ cells. A 1-to-1 ratio of both cell types is preferred since it provides synergistic antitumor effects.

CAR T-cells are engineered to transfer arbitrary specificity onto an immune effector cell, like a T cell, which specifically eliminates antigen-bearing tumor cells. The CAR may comprise a scFv being derived from an antibody, a CD3ζ and a transmembrane domain (so-called first-generation CARs). In this way, the engineered CAR is able to recognize specific tumor associated-antigens. Therefore, the CAR has the ability to bind unprocessed tumor surface antigens without MHC processing while TCRs engage with both tumor intracellular and surface antigenic peptides embedded in MHC.

In contrast, TCRs are α/β heterodimers that bind to the MHC-bound antigens. As discussed above, CARs recognize tumor antigen which led to T-cell activation with different functions compared with TCR. CAR-T cell therapy has certain disadvantages like off-tumor toxicities when targeting tumor-specific antigen. Compared with CARs, TCRs have several structural advantages in T cell-based therapy, such as more subunits in their receptor structure (ten subunits vs one subunit), greater immunoreceptor tyrosine-based activation motif (ITAMs) (ten vs three), less dependence on antigens (one vs 100), and more co-stimulate receptors (CD3, CD4, CD28, etc.) (Zhao et al. (2021) Front. Immunol.,|https://doi.org/10.3389/fimmu.2021.658753).

CAR NK-cells are distinguished from CAR T-cells in that the chimeric antigen receptor is introduced into NK cells instead of T-cells. Just as CAR T-cells, CAR-NK cells can be engineered to target diverse antigens, enhance proliferation and persistence in vivo, increase infiltration into solid tumours, overcome resistant tumour microenvironment, and ultimately achieve an effective anti-tumour response.

Natural cytotoxicity receptors NK cells (NCR NK-cells) are NK-cell that have been genetically engineered to express a NCR. The NCRs have been proposed to bind to many cellular ligands which are implicated in NK cell surveillance of tumor cells. Many of these interactions have been shown to evoke the cytotoxic and cytokine-secreting functions of NK cells. However, it is also possible that the NCRs may regulate other anti-tumor pathways. NCRs and their ligands can be successfully targeted for cancer immunotherapy. NCRs have been classically defined as activating receptors delivering potent signals to NK cells in order to lyse harmful cells and to produce inflammatory cytokines.

TCR/CAR hybrid T-cells are T-cells that have been genetically engineered to express a TCR and CAR. Similarly, NCR/CAR hybrid NK-cells are T-cells that have been genetically engineered to express a NCR and CAR.

Tumor-infiltrating lymphocytes (TILs) are white blood cells that have left the bloodstream and migrated towards a tumor. TILs are implicated in killing tumor cells. The presence of lymphocytes in tumors is often associated with better clinical outcomes.

In accordance with a more preferred embodiment of the invention, the tumor-infiltrating lymphocytes are tumor-infiltrating T-cells or tumor-infiltrating NK cells.

In adoptive T-cell transfer therapy, TILs are expanded ex vivo from surgically resected tumors that have been cut into small fragments or from single cell suspensions isolated from the tumor fragments. Multiple individual cultures are established, grown separately and assayed for specific tumor recognition. TILs are typically expanded over the course of a few weeks with a high dose of IL-2 in 24-well plates. Selected TIL lines that presented best tumor reactivity are then further expanded in a “rapid expansion protocol” (REP), which uses anti-CD3 activation for a typical period of two weeks. The final post-REP TIL is infused back into a patient in order to treat a tumor of the patient. This applies mutatis mutandis to adoptive NK-cell transfer with TILs.

In accordance with a further preferred embodiment of the invention the anti-tumor lymphocytes are autologous anti-tumor lymphocytes.

In an anti-tumor therapy with autologous lymphocytes the lymphocytes are taken from a subject having a tumor and are genetically engineered (e.g. to produce CAR T-cells) and/or selected and/or expanded (e.g. to produce TILs) ex vivo and then transferred back into the same subject. These autologous therapies are subject-specific because the therapeutic cells are created from a subject's own cells.

In accordance with another preferred embodiment of the invention the arginine at amino acid position 255 is substituted.

As discussed herein above, variants of cGAS wherein arginine at amino acid position 255 is substituted have been observed to produce more cGAMP as compared to the variant cGAS wherein arginine at amino acid position 236 is substituted. The one or both arginines at amino acid positions 255 and 236 therefore preferably comprise(s) and most preferably reflect arginine at amino acid position 255.

In accordance with a related preferred embodiment of the invention the one or both arginines at amino acid positions 255 and 236 and/or the one or both lysines at amino acid positions 254 and 258 is/are substituted by a non-conservative amino acid, preferably by an amino acid with a hydrophobic side chain and most preferably by alanine or glutamic acid.

As discussed herein above, human wild-type cGAS can be activated by replacing one or both arginines at amino acid positions 255 and 236 by glutamic acid or arginine. It is furthermore believed that the same technical effect can be achieved by an amino acid substitution of one or both lysines at amino acid positions 254 and 258 by glutamic acid or arginine.

Since arginine and lysine are basic amino acids while alanine is an amino acid with a hydrophobic side chain (or hydrophobic amino acid) and glutamic acid is an acidic amino acid, the substitution of arginine or lysine by alanine is in accordance with the above preferred embodiment a non-conservative amino acid substitution.

A non-conservative amino acid substitution of arginine or lysine is preferably the substitution of arginine with another naturally occurring amino acid other than the two basic amino acids histidine and lysine/arginine, respectively, preferably an amino acid with a hydrophobic side chain or an acidic amino acid. The amino acid with a hydrophobic side chain is preferably selected from alanine, valine, methionine, leucine, isoleucine, proline, tryptophan and phenylalanine. The acidic amino acid is preferably selected from glutamic acid and aspartic acid.

In accordance with a preferred embodiment of the invention the tumor is cancer, preferably a solid cancer.

Cancer is an abnormal malignant growth of tissue that possesses no physiological function and arises from uncontrolled usually rapid cellular proliferation. The cancer is preferably selected from the group consisting of breast cancer, ovarian cancer, endometrial cancer, vaginal cancer, vulva cancer, bladder cancer, salivary gland cancer, pancreatic cancer, thyroid cancer, kidney cancer, lung cancer, cancer concerning the upper gastrointestinal tract, colon cancer, colorectal cancer, prostate cancer, squamous-cell carcinoma of the head and neck, cervical cancer, glioblastomas, malignant ascites, lymphomas and leukemias. Among this list of cancers gynecologic cancers (i.e. cervical, ovarian, uterine, vaginal, and vulvar cancer) are preferred and breast cancer and ovarian cancer are most preferred. Also preferred among this list of cancers is bladder cancer.

The tumor or cancer is preferably a solid tumor or cancer. A solid tumor or cancer is an abnormal mass of tissue that usually does not contain cysts or liquid areas by contrast to a liquid tumor.

In accordance with a preferred embodiment of the invention the subject is human.

The subject may be a subject being at risk of developing a tumor and is preferably a subject wherein a tumor has been diagnosed.

The subject may receive a further anti-tumor treatment before, along with or after the medical use of the invention. Non-limiting examples of such anti-tumor treatments are surgery, radiation therapy and chemotherapy.

In accordance with a preferred embodiment of the invention the variant cyclic GMP-AMP synthase (cGAS) carrying an amino acid substitution of one or both arginines at amino acid positions 255 or 236 and/or the one or both lysines at amino acid positions 254 and 258 comprises of consists of (a) an amino acid sequence selected from any one of SEQ ID NOs 1 to 3 and SEQ ID NOS 12 to 23, or (b) an amino acid sequence sharing at least 80%, preferably at least 90% identity with SEQ ID NO: 4, provided that one or both arginines at amino acid positions 255 or 236 and/or the one or both lysines at amino acid positions 254 and 258 are substituted by another amino acid; (c) the amino acid sequence of (a) or (b) further comprising an nuclear localization sequence (NLS), or (d) the amino acid sequence of any one of (a) to (c), wherein a part of or the complete IDR is deleted; and/or wherein the nucleic acid molecule encoding said variant cGAS comprises of consists of (c) a nucleotide sequence selected from any one of SEQ ID NOs 5 to 7 an SEQ ID NOs 24 to 35, or (d) a nucleotide sequence sharing at least 80%, preferably at least 90% identity with SEQ ID NO: 8, provided that the base triplet(s) encoding one or both arginines at amino acid positions 255 or 236 and/or one or both lysines at amino acid positions 254 and 258 are substituted by another amino acid; (c) the nucleotide sequence or (a) or (b) further encoding a nuclear localization sequence (NLS), or (d) the nucleotide sequence of any one of (a) to (c), wherein a part of or the complete nucleotide sequence encoding the IDR is deleted.

SEQ ID NOs 1 to 3 are the amino acid sequence of human cGAS R255A, R236A and R255A/R236A. SEQ ID NOS 12 to 23 are the amino acid sequence of human cGAS R254A, R258A and all possible combination of at least two, at least three and all four of R255A, R236A, R254A and R258A with the exception of the double mutant R255A/R236A. Among SEQ ID NOs 1 to 3 and SEQ ID NOs 12 to 23, SEQ ID NOS 1 to 3 are preferred, and SEQ ID NOs 1 and 3 are most preferred. SEQ ID NO: 4 is the full-length amino acid sequence of wild-type human cGAS. SEQ ID NOs 5 to 7 are the nucleic acid sequences encoding the amino acid sequence of human cGAS R255A, R236A and R255A/R236A. The nucleic acid sequences of SEQ ID NOs 24 to 35 encode the amino acid sequences of SEQ ID NOs 12 to 23. Among SEQ ID NOs 5 to 7 and SEQ ID NOs 24 to 35, SEQ ID NOs 5 to 7 are preferred, and SEQ ID NOs 5 and 7 are most preferred. SEQ ID NO: 8 is the nucleic acid sequences encoding the amino acid sequence of the full-length wild-type human cGAS.

The sequence sharing of at least 80% identity is with increasing preference at least 80%, at least 85%, at least 90%, at least 95%, at least 96%, at least 97%, at least 98%, and at least 99% identical.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment, for two or more sequences or subsequences the percentage of amino acid residues or nucleotides that are the same (e.g. 70%, 75%, 80%, 85%, 90% or 95% identity) may be determined, when the (sub) sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schäffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences. The NCBI BLAST algorithm is available for protein (Protein BLAST) and nucleotides (Nucleotide BLAST). For Protein BLAST the algorithm parameters are preferably: max target sequences: 100, with automatically adjust parameters for short input sequences, expect threshold 0.05, word size 6, Max matches in a query range 0, matrix BLOSUM62, cap cost existence: 10 extension: 1, and compositional adjustment. For Nucleotide BLAST the algorithm parameters are preferably: max target sequences: 100, with automatically adjust parameters for short input sequences, Expect threshold 0.05, word size 28, Max matches in a query range 0, match/mismatch scores 1,-2, cap costs linear, low complexity regions filter, and, ask for look up table only. These are the standard algorithm parameters for protein BLAST and Nucleotide BLAST and they can adjusted, if needed.

The nuclear localization sequence (NLS) is an amino acid sequence that ‘tags’ a protein for import into the cell nucleus by nuclear transport. Typically, this signal consists of one or more short sequences of positively charged lysines or arginines exposed on the protein surface.

NLS can be classified as either monopartite or bipartite. The major structural differences between the two are that the two basic amino acid clusters in bipartite NLSs that are separated by a relatively short spacer sequence (hence bipartite—2 parts), while monopartite NLSs are not separated. The first NLS to be discovered is the sequence PKKKRKV (SEQ ID NO: 9) in the SV40 Large T-antigen (a monopartite NLS) which is encoded by SEQ ID NO: 10. The SV40 Large T-antigen was also used in the appended examples as NLS. The NLS of nucleoplasmin, KRPAATKKAGQAKKKK (SEQ ID NO: 11), is the prototype of the ubiquitous bipartite signal: two clusters of basic amino acids, separated by a spacer of about 10 amino acids. Both signals are recognized by importin α. Importin α contains a bipartite NLS itself, which is specifically recognized by importin β. The latter can be considered the actual import mediator.

The intrinsically disordered region (IDR) region can be found at the N-terminus of the full-length human cGAS and spans with increasing preference the amino acid positions corresponding to amino acid positions 1-157, amino acid positions 1-155 and amino acid positions 1-146 of the full-length human cGAS. As discussed above, the amino acid sequence of the full length wild-type human cGAS is shown in SEQ ID NO: 4 and this amino acid sequence is encoded by SEQ ID NO: 8.

In accordance with a yet further preferred embodiment of the invention, the nucleic acid molecule encoding said variant cGAS is RNA or DNA.

The RNA is preferably mRNA. The mRNA may be introduced into a cell, wherein the host cell is preferably an anti-tumor lymphocyte according to the invention. Means and methods for introducing the mRNA into the genome of a host cell will be discussed herein below, in particular in connection with lipid nanoparticles.

In this connection it is of note that the host cells as used in the examples are cells wherein the wild-type cGAS has been knocked-out. However, this is not an essential feature because and as explained above the wild-type cGAS is inactive in the host cells and only the variant cGAS is active: i.e results in the production of cGAMP with the host cell. Hence, the host cells may be wild-type host cells or host cells harboring wild-type cGAS.

The DNA is preferably genomic DNA or cDNA comprises in an expression vector. The genomic DNA and expression vector will be further described herein below.

The nucleic acid molecule encoding said variant cGAS may preferably and generally be formulated as vesicles, such as liposomes or exososmes. Liposomes have attracted great interest because of their specificity and the duration of action they offer from the standpoint of drug delivery. Liposomal cell-type delivery systems have been used to effectively deliver nucleic acids, such as siRNA in vivo into cells (Zimmermann et al. (2006) Nature, 441:111-114). Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are phagocytosed by macrophages and other cells in vivo. Exosomes are lipid packages which can carry a variety of different molecules including RNA (Alexander et al. (2015), Nat Commun; 6:7321). The exosomes including the molecules comprised therein can be taken up by recipient cells. Hence, exosomes are important mediators of intercellular communication and regulators of the cellular niche. Exosomes are useful for diagnostic and therapeutic purposes, since they can be used as delivery vehicles, e.g. also for contrast agents or drugs.

In accordance with a yet further preferred embodiment of the invention the nucleic acid molecule encoding said variant cGAS is comprised in lipid nanoparticles (LNP), wherein the LNPs were preferably contacted with the anti-tumor lymphocytes prior to their use in the treatment of a tumor in a subject, so that the nucleic acid molecule encoding said variant cGAS and the LNPs were released into the cytoplasm of the anti-tumor lymphocytes.

Lipid nanoparticles have been developed as vehicles for small molecule delivery by the nanomedicine and materials communities and are now a key component of COVID-19 mRNA vaccines. Lipid nanoparticles are particularly useful as mRNA carriers.

Lipid nanoparticles are spherical vesicles made of ionizable lipids, which are positively charged at low pH (enabling RNA complexation) and neutral at physiological pH (reducing potential toxic effects, as compared with positively charged lipids, such as liposomes). Owing to their size and properties, lipid nanoparticles are taken up by cells via endocytosis, and the ionizability of the lipids at low pH (likely) enables endosomal escape, which allows release of the cargo into the cytoplasm. In addition, lipid nanoparticles usually contain a helper lipid to promote cell binding, cholesterol to fill the gaps between the lipids, and a polyethylene glycol (PEG) to reduce opsonization by serum proteins and reticuloendothelial clearance. The relative amounts of ionizable lipid, helper lipid, cholesterol and PEG substantially affect the efficacy of lipid nanoparticles, and need to be optimized for a given application and administration route. Moreover, lipid type, size and surface charge impact the behaviour of lipid nanoparticles in vivo.

Hence, in the preferred case of contacting the LNPs with the anti-tumor lymphocytes prior to their use in the treatment of a tumor in a subject, the LNPs are taken up by anti-tumor lymphocytes via endocytosis and the nucleic acid molecule encoding said variant cGAS are released into the cytoplasm of the anti-tumor lymphocytes.

In accordance with another preferred embodiment of the invention the nucleic acid molecule encoding said variant cGAS is an expression vector, preferably an expression vector within the anti-tumor lymphocytes.

The term “expression vector” in accordance with the invention means preferably a plasmid, cosmid, virus, bacteriophage or another vector used e.g. conventionally in genetic engineering which carries the nucleic acid molecule of the invention in an expressible form. The expressible form is preferably a form, wherein the expression can be controlled, such as a Tet-on or Tet-off system. Here, the expression can be turned on and off by tetracycline or doxycycline. A Tet-on system and the use of doxycycline are illustrated in the examples.

The nucleic acid molecule of the invention may, for example, be inserted into several commercially available expression vectors. Non-limiting examples include lentiviral expression vector and prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), PEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecules inserted into the vector can e.g. be synthesized by standard methods, or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide encoding the polypeptide/protein or fusion protein of the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycine, and kanamycin. Specifically-designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded peptide or fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

In accordance with a further preferred embodiment of the invention the nucleic acid molecule encoding said variant cGAS is within the genome of the anti-tumor lymphocytes.

In the case of genomic DNA the nucleic acid molecule encoding said variant cGAS is part of the genome of a host cell. The term “host cell” means in this connection any cell of any organism that is selected, modified, transformed, grown, used or manipulated in any way, for the production of the variant cGAS according to the invention by the cell.

The host cell is in accordance with the above preferred embodiment anti-tumor lymphocyte according to the invention. Means and methods for introducing the nucleic acid molecule encoding said variant cGAS into the genome of a host cell will be discussed herein below.

In accordance with a more preferred embodiment of the invention the nucleic acid molecule encoding said variant cGAS has been inserted into the genome of the anti-tumor lymphocytes by a genome editing technology, such as meganuclease, Zn-finger, TALEN or CRISPR.

The nucleic acid molecule encoding the variant cGAS may be introduced into the genome of the anti-tumor lymphocytes by homologous recombination. The homologous recombination is preferably Cre-Lox recombination. This is a site-specific recombinase technology, used to carry out deletions, insertions, translocations and inversions at specific sites in the DNA of cells. It allows the DNA modification to be targeted to a specific cell type or be triggered by a specific external stimulus. It is implemented both in eukaryotic and prokaryotic systems.

Over the past years homologous recombination has been largely replaced by genome editing technologies, such as meganuclease, Zn-finger, TALEN or CRISPR. The genome editing technology is preferably CRISPR.

Meganucleases are enzymes in the endonuclease family which are characterized by their capacity to recognize and cut large DNA sequences (from 12 to 40 base pairs). The most widespread and best known meganucleases are the proteins in the LAGLIDADG family, which owe their name to a conserved amino acid sequence. Meganucleases, found commonly in microbial species, have the unique property of having very long recognition sequences (>14 bp) thus making them naturally very specific. In order to find the exact meganuclease required to act on a specific DNA sequence, mutagenesis and high throughput screening methods are available to create a meganuclease variant that recognizes a desired target sequences. It is also possible to fuse meganucleases to each other, thereby creating hybrid enzymes that recognize a new sequence. Moreover, a method named rationally designed meganuclease (U.S. Pat. No. 8,021,867) may be used to design sequence specific meganucleases.

The concept behind ZFNs and TALEN technology is based on a non-specific DNA cutting enzyme, which can then be linked to specific DNA sequence recognizing peptides such as zinc fingers and transcription activator-like effectors (TALEs). The key to this was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity.

Zinc finger motifs occur in several transcription factors. The zinc ion, found in 8% of all human proteins, plays an important role in the organization of their three-dimensional structure. In transcription factors, it is most often located at the protein-DNA interaction sites, where it stabilizes the motif. The C-terminal part of each finger is responsible for the specific recognition of the DNA sequence. The recognized sequences are short, made up of around 3 base pairs, but by combining 6 to 8 zinc fingers whose recognition sites have been characterized, it is possible to obtain specific proteins for sequences of around 20 base pairs. It is therefore possible to control the expression of a specific gene. The method generally adopted for this involves associating two proteins—each containing 3 to 6 specifically chosen zinc fingers—with the catalytic domain of the Fokl endonuclease. The two proteins recognize two DNA sequences that are a few nucleotides apart. Linking the two zinc finger proteins to their respective sequences brings the two endonucleases associated with them closer together. Fokl requires dimerization to have nuclease activity and this means the specificity increases dramatically as each nuclease partner would recognize a unique DNA sequence. To enhance this effect, Fokl nucleases have been engineered that can only function as heterodimers and have increased catalytic activity.

Transcription activator-like effector nucleases (TALENS or TAL nucleases) are artificial restriction enzymes generated by fusing a specific DNA-binding domain to a non-specific DNA cleaving domain. The DNA binding domains, which can be designed to bind any desired DNA sequence, comes from TAL effectors, DNA-binding proteins excreted by plant pathogenic Xanthomanos sp. Tal effectors consists of repeated domains, each which contains a highly considered sequence of 34 amino acids, and recognize a single DNA nucleotide. The nuclease can create double strand breaks at the target site that can be repaired by error-prone non-homologous end-joining (NHEJ), resulting in gene disruptions through the introduction of small insertions or deletions. TALEN constructs are used in a similar way to designed zinc finger nucleases, and have at least three advantages in targeted mutagenesis: (1) DNA binding specificity is higher, (2) off-target effects are lower, and (3) construction of DNA-binding domains is easier.

As is evident from the above, in meganucleases, ZNF nucleases, and TAL nucleases the endonuclease activity and the site-specificity within the target genome are conferred by one compound.

The CRISPR or CRISPR-Cas genome editing system was adapted from a naturally occurring defense system against foreign DNA (e.g. viruses, plasmid DNA) in prokaryotes. Prokaryotes with CRISPR-Cas system capture fragments of DNA from invading DNA and integrate them into DNA segments known as CRISPR arrays. The CRISPR arrays allow the bacteria/archaea to acquire immunity against the invading DNA (or homologous ones). The bacteria/archaea produce CRISPR-RNAs (crRNAs) from the CRISPR arrays to target the foreign DNA, which in complex with CRISPR nucleases (e.g. Cas9 or a similar enzyme) inactivate the invading DNA by nucleolytic cleavage.

The CRISPR-Cas system has been harnessed for genome editing in prokaryotes and eukaryotes. A small piece of RNA with a short “guide” sequence that attaches (binds) to a specific target sequence of DNA in a genome is created (the so-called guide RNA (gRNA) or single guide (sgRNA)). The genomic target site of the gRNA can be any ˜20 nucleotide DNA sequence, provided it meets two conditions: (i) The sequence is unique compared to the rest of the genome, and (ii) the target is present immediately adjacent to a Protospacer Adjacent Motif (PAM). The PAM sequence is essential for target binding, but the exact sequence depends on which CRISPR endonuclease is used. CRISPR endonuclease and their respective PAM sequences are known in the art (see https://www.addgene.org/crispr/guide/#pam-table). Hence, the gRNA also binds to the CRISPR endonuclease (e.g. the Cas9 or Cpf1 enzyme). As in bacteria, the gRNA is used to recognize the DNA sequence, and the CRISPR endonuclease cuts the DNA at the targeted location. Once the DNA is cut, the cell's own DNA repair machinery (NHEJ or HDR) adds or deletes pieces of genetic material, or makes changes to the DNA by replacing an existing segment with a customized DNA sequence. Hence, in the CRISPR-Cas system, the CRISPR nuclease makes a double-stranded break in DNA at a site determined by the short (˜20 nucleotide) gRNA which break is then repaired within the cell by NHEJ or HDR. The CRISPR-Cas system can be multiplexed by adding multiple gRNAs. It was demonstrated that, for example, five different simultaneous mutations can be introduced into mouse embryonic stem cells by using five different gRNA molecules and one CRISPR endonuclease.

As is evident from the above, in the CRISPR technology the endonuclease activity (CRISPR nuclease) and the site-specificity within the target genome (gRNA) are conferred by two separate compounds.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

FIG. 1|Expression of cGAS R241E variant in murine 4T1 breast cancer cells. a, 4T1 cells knocked out for cGAS and STING and transduced with lentiviruses encoding for doxycycline (Dox)-inducible cGAS^{WT} and cGAS^{R241E} variant, respectively, were incubated with Dox (1 μg/ml) for 72 h and cGAS protein levels were determined by western blot. b, c, 4T1 wild-type cells, 4T1 cGAS knockout (KO) cells, and 4T1 cGAS KO reconstituted with dox-inducible cGAS^{WT} and cGAS^{R241E} variant, respectively, were incubated with Dox (1 μg/ml) for 48 h and relative production of cGAMP was assessed in the cell supernatant (b) and in the cell lysate (c). d, Cells as described in (b, c) were treated with Dox (1 μg/ml) continuously for 14 days and cell viability was assessed by MTS assay. e, Assessment of STING protein levels in pancreatic ductal adenocarcinoma (PDAC) cells, 4T1 cells, E0771 breast cancer cells, and CT26 cells.

FIG. 2|Effect of intratumoral cGAS R241E variant expression on tumor growth and immune cell activation. a, Workflow of in vivo studies. b, c, Average (b) and individual (c) tumor measurements of BALB/c mice (n=9 per group) injected with cGAS KO 4T1 cells reconstituted with Dox-inducible cGAS^{WT} and cGAS^{R241E} variant, respectively. Dox was administered via the drinking water from day 9 to day 21. For cGAS^{WT} expressing tumors, n=1 out of n=9 tumors regressed completely and for CGAS^{R241E} expressing tumors n=6 out of n=9 tumors regressed completely. d, Relative frequencies of M1 macrophages (CD11b⁺Ly6CGC⁻CD206⁻F4/80⁺Ly6C⁺MHCII⁺), DCs (CD11b⁺Ly6CGC⁻CD206⁻F4/80⁺Ly6C⁻MHCII⁺), CD68^{high} NK cells (TCRb⁻NKp46⁺CD69^{hi}), and CD4 positive T cells (CD3⁺TCRb⁺CD4⁺) in cGAS^{WT} (n=2 mice) and cGAS^{R241E} (n=3 mice) variant expressing tumors. e, Relative frequencies of CD8+ T cells, CD3+ lymphocytes, and CD4+ Helper T cells in the spleens of mice bearing cGAS^{WT} (n=6 mice) and cGAS^{R241E} (n=6 mice) variant expressing tumors. f, Relative frequency of CD4+helper T cells in the draining lymphnodes of mice bearing cGAS^{WT} (n=6 mice) and cGAS^{R241E} (n=6 mice) variant expressing tumors.

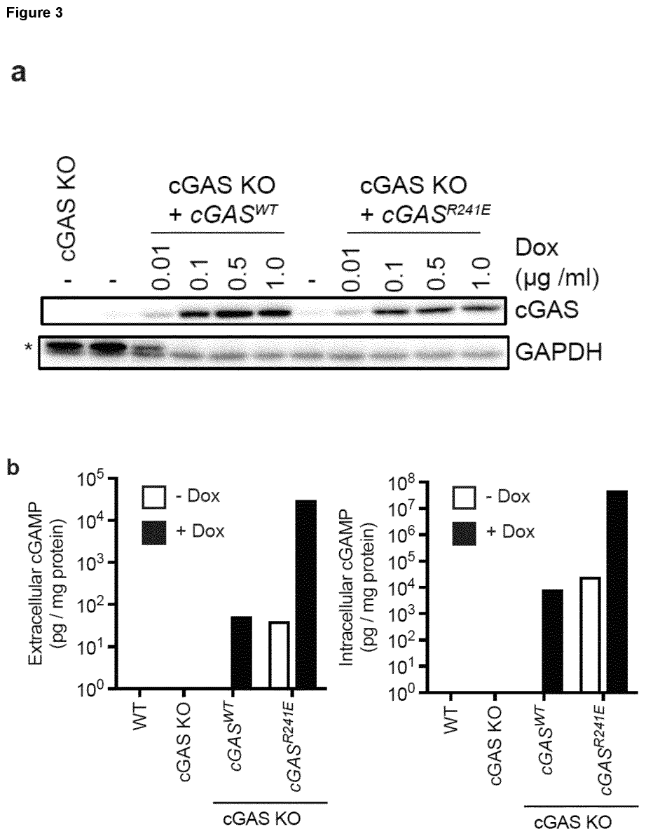

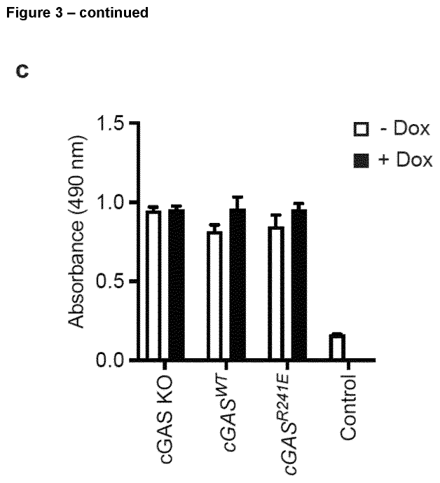

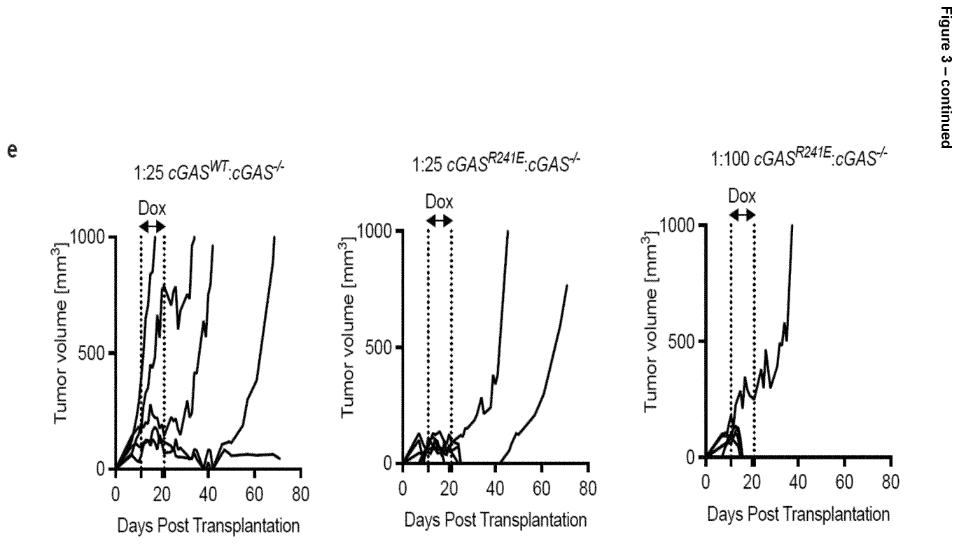

FIG. 3|Assessment of cGAS R241E variant expression in CT26 colorectal cancer cells in vitro and in vivo. a, CT26 cells knocked out or cGAS and transduced with lentiviruses encoding for doxycycline (Dox)-inducible cGAS^{WT} and cGAS^{R241E} variant, respectively, were incubated with Dox (concentration as indicated) for 72 h and cGAS protein levels were determined by western blot. b, CT26 wild-type cells, CT26 cGAS knockout (KO) cells, and CT26 cGAS KO cells reconstituted with dox-inducible cGAS^{WT} and cGAS^{R241E} variant, respectively, were incubated with Dox (1 μg/ml) for 48 h and relative production of cGAMP was assessed in the cell supernatant (left panel) and in the cell lysate (right panel). c, Cells as described in (b) were treated with Dox (1 μg/ml) continuously for 10 days and cell viability was assessed by MTS assay. d, Tumor measurements of CT26 cGAS knockout (KO) cells and CT26 cGAS KO cells reconstituted with dox-inducible cGAS^{WT} and cGAS^{R241E} variant, respectively, transplanted into BALB/c mice (n=5 mice per group) are shown. e, CT26 cGAS KO reconstituted with CGAS^{WT} and cGAS^{R241E} were mixed with CT26 cGAS KO at a ratio of 1:25 (both) and 1:100 (only cGAS^{R241E}) and transplanted into BALB/c mice (n=5 mice per group). Dox was administered from day 11 to day 21 after transplantation and tumor measurements of individual mice are shown. For tumors containing 1:25 cGAS^{WT}:cGAS KO expressing tumors, n=1 out of n=5 tumors regressed completely and for each mixture of 1:25 and 1:100 cGAS^{R241E}: cGAS KO expressing tumors n=4 out of n=5 tumors regressed completely.

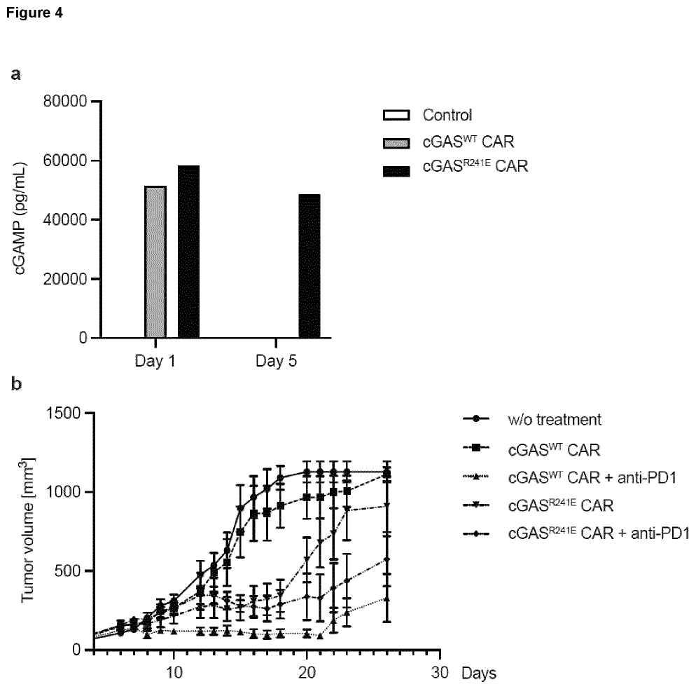

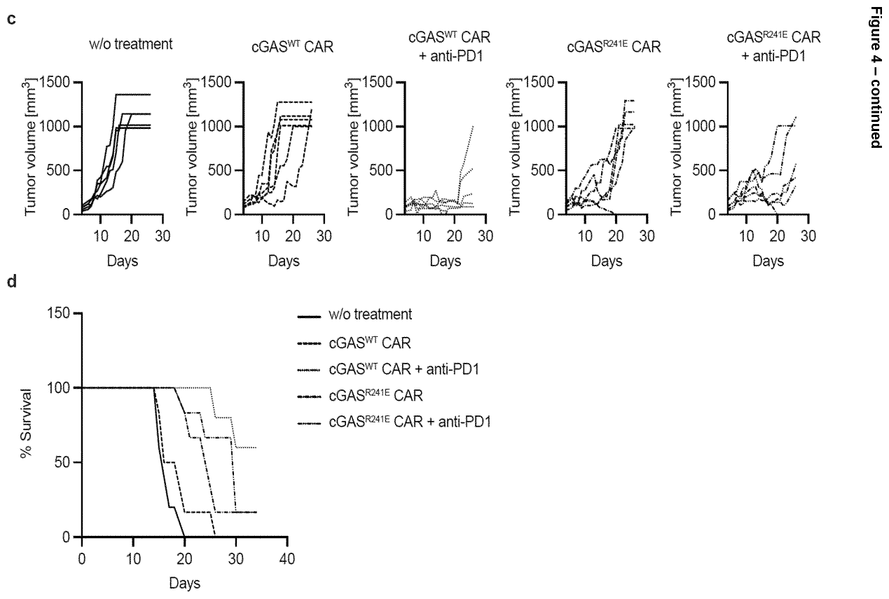

FIG. 4|Characterization of the antitumor potential of cGAS functionalized CAR T cells. a, cGAMP production in the supernatant of mouse T cells from Sting1 deficient mice one or five days following retroviral transduction with constructs expressing a cGAS^{WT} or cGAS^{R241E} variant in conjunction with a construct encoding for a HER2 targeting CAR (cGAS^{WT} CAR and cGAS^{R241E} CAR, respectively). b, c, Tumor growth (mean curves shown in (b) and individual curves shown in (c)) in mice bearing MC38-HER2 expressing tumors. Treatment groups were as follows: non-treated (w/o treatment), cGAS^{WT} CAR and cGAS^{R241E} CAR alone or in combination with anti-PD1, respectively. d, Survival curves of the mice shown in (b, c).

### THE EXAMPLES ILLUSTRATE THE INVENTION

### Example 1

### cGAS Super-Enzyme Expression Reduces Tumor Burden in Pre-Clinical Models of Cancer

Manipulation of the innate immune system holds great promise as a means to target tumor cells more efficiently. Most research has focused on defining principals on how to engage activating receptors or pathways that elicit powerful antitumor immunity. By contrast, there is little information on how to exploit negative innate immune “checkpoints” as a therapeutic avenue against cancer.

Recently, a fundamental mechanism of negative regulation of the innate immune sensor, cyclic GMP-AMP synthase (cGAS), has been discovered. Specifically, it was found that cGAS is anchored to the nucleosome core particle so to prevent the binding of genomic self-DNA and avoid autoreactivity. In the following research, it was asked whether mutation in a residue that is essential for nucleosome-cGAS interaction can promote immune activation and tumor clearance, if expressed inside the tumor environment.

To study the role of cGAS super-enzymes in tumor suppression, cGAS knockout (KO) murine mammary carcinoma 4T1 cells and murine colorectal carcinoma CT26 cells were engineered and subsequently transduced the cells with a lentivirus encoding for doxycycline-inducible murine wild-type (WT) cGAS (4T1^{WT} cells or CT26^{WT} cells) or the cGAS variant encoding for the R241E substitution (4T1^{R241E} cells or CT26^{R241E} cells). Focusing on 4T1 cells as a model for poorly immunogenic tumors, it was confirmed that 4T1WT cells and 4T1^{R241E} cells responded to doxycycline (Dox) treatment with cGAS expression (FIG. 1a). Moreover, probing cell supernatants by ELISA was determined that induced expression of cGAS led to the secretion of cGAMP from 4T1^{R241E}, whereas no extracellular cGAMP was detected in supernatants from 4T1^{WT} cells (FIG. 1b). By contrast, intracellular cGAMP was detected in lysates of both 4T1^{WT} cells and 4T1^{R241E} cells, albeit levels in lysates from 4T1^{R241E} cells were much higher compared to 4T1^{WT} cells (FIG. 1c). It was also noted that 4T1^{R241E} cells synthesized intracellular cGAMP even in the absence of doxycycline treatment, likely as a result from the leakiness of the dox-inducible system used in this study (FIG. 1c). No appreciable difference in cytotoxicity was observed in the distinct engineered 4T1 cells lines in the presence or absence of dox treatment, as assessed by MTS assay (FIG. 1d). Importantly, assessing STING expression across distinct tumor cell lines, it was noted that some cell lines, including parental 4T1 cells, but not CT26 cells, lacked expression of STING, which accords with the heterogenous expression of STING in tumor cell lines as observed by others (Xia et al., 2016) (FIG. 1e). Therefore, biological effects emanating from dox-induced cGAS expression in 4T1^{WT} cells or 4T1^{R241E} cells can solely be ascribed cell-extrinsic effects of cGAMP, but not to STING-dependent transcriptional or non-transcriptional cell-intrinsic activities.

It was next tested the effect of cGAS super-enzyme variant on tumor growth in vivo. To this end, 4T1 cells were implanted into the mammary fad pad of BALB/c mice and, after tumor establishment, dox was administered to induce cGAS expression for 11 days (FIG. 2a). Following dox treatment, 4T1^{WT} tumors showed a partial initial response, but all tumors ultimately grew until the defined endpoint (FIG. 2b). By contrast, 4T1^{R241E} tumors displayed a much stronger initial response and 6 out of 9 tumors completely disappeared until the end of the observation period (FIG. 2b). To assess the involvement of an immune response, immune cells were profiled in the tumor by cytometry by time of flight (CyTOF) and immune cells in the spleen and draining lymph nodes flow cytometry analysis 15 days after transplantation and 6 days after dox treatment (FIG. 2a). CyTOF analysis showed that compared to cGAS^{WT} expression of cGAS^{R241E} resulted in higher frequencies of M1 polarized macrophages, dendritic cells, CD68^{high} NK cells, and CD4 positive T cells in the tumor (FIG. 2d). Further, 4T1 tumors expression CGAS^{R241E} displayed significantly increased frequency of CD3, CD4, and CD8 positive T lymphocytes in the spleen and increased frequency of CD4 positive T lymphocytes in the draining lymph nodes relative to cGAS^{WT} expressing 4T1 tumors (FIG. 2e, f). Together, these data show that the expression of the cGAS R241E variant promotes tumor regression in vivo along with inducing an immune response.

Next the effect of cGAS variants in an immunogenic tumor model was examined based on CT26 cell transplantation. Following lentiviral transduction, CT26 cells responded to Dox treatment with inducible GAS expression (FIG. 3a). However, it was noticed that subtle cGAS expression was already detected in the absence of doxycycline treatment, likely due to the leakiness of the inducible expression system. This limited level of cGAS expression in the absence of doxycycline was sufficient to trigger intracellular production and extracellular release of cGAMP from CT26^{R241E} cells, but not from CT26^{WT} cells (FIG. 3b). In the presence of Dox CT26^{R241E} cells produced more cGAMP compared to CT26^{WT} cells (FIG. 3b). CT26^{WT} cells and CT26^{R241E} cells grew similar and no obvious cytopathic effect was observed upon Dox treatment (FIG. 3c). When transplanted in BALB/c mice CT26^{WT} cells, but not CT26^{R241E} cells generated tumors in the absence of Dox administration (FIG. 3d). This latter observation suggests that constitutive cGAMP production by CT26^{R241E} cells interferes with the establishment of tumors in vivo. Next mixed cancer cell populations comprising parental CT26 cGAS KO cells and distinct ratios of CT26^{WT} cells or CT26^{R241E} cells, respectively, were used for in vivo studies. It was found that diluting CT26^{R241E} cells with CT26 cGAS KO cells generated subcutaneous tumors (FIG. 3e). Following doxycycline administration, 4 out of 5 tumors completely regressed at ratios of 1:25 and 1:100 CT26^{R241E} to CT26 cGAS KO cells (FIG. 3e). By contrast, only 1 out of 5 tumors regressed when CT26^{WT} cells were used at a 1:25 ratio together with CT26 cGAS KO cells (FIG. 3e). Together, these data show that the expression of cGAS variant in a minority of cells within tumors is sufficient to promote the rejection of established tumors.

In summary, it can be concluded from these studies that expression of cGAS variant in tumors in vivo can promote efficient tumor eradication. Analyzing the immune cell population in tumors, lymph nodes, and spleen, further indicates to the activation of a powerful immune program induced by the expression of the cGAS variants that entails the activation of both innate and adaptive immune cells. Finally, our data from immunogenic CT-26 tumor cells, provides a proof-of-concept that expression of the cGAS variant in a subset of tumors is sufficient to trigger effective tumor eradication. In sum, these examples demonstrated the utility of cGAS variants as a novel tool immunotherapy concept for more efficient cancer therapies.

### Example 2

### Efficacy of cGAS Functionalized CAR T Cells in Cancer Bearing Mice

To translate the antitumor potential of cGAS super-enzymes into a clinical setting, T cells with viral vectors were stably transduced encoding for cGAS^{WT} or cGAS^{R241E} and a chimeric antigen receptor (CAR) construct targeting HER2. To rule out T cell intrinsic engagement of STING signaling, T cells from Sting1 deficient mice were used for the consecutive experiments. Measuring extracellular cGAMP levels revealed that shortly after transduction (one day) both cGAS^{WT} and cGAS^{R241E} CAR T cells showed significant cGAMP production (FIG. 4a). By contrast, after five days notable cGAMP production was only apparent in the supernatant collected from cGAS^{R241E} CAR T cells, but not from cGAS^{WT} CAR T cells (FIG. 4a).

Next, the antitumor potential of cGAS functionalized CAR T cells was assessed against established tumors in vivo. To this end, HER2-expressing MC38 colorectal cancer cells were generated and transplanted into wild-type mice via subcutaneous injection. Once tumors were palpable, mice were randomly assigned to receive no treatment (w/o treatment), cGAS^{WT} CAR T cells or cGAS^{R241E} CAR T cells. Additionally, some CAR T cell receiving mice were treated with anti-PD1. It was observed that CGAS^{R241}E CAR T cells potently inhibited tumor growth (FIG. 4b, c). By contrast, tumor growth between non-treated mice and those receiving cGAS^{WT} CAR T cells was similar (FIG. 4b, c). Reduced tumor growth was further noticed in mice receiving either cGAS^{WT} CAR T cells or cGAS^{R241E} CAR T cells in combination with anti-PD1 (FIG. 4b, c). Accordingly, a survival benefit was observed for tumor bearing mice treated with cGAS^{R241E} CAR T cells alone or in combination with anti-PD1 and cGAS^{WT} CAR T cells combined with anti-PD1. Taken together, these results demonstrate that cGAS functionalized CAR T cells are effective in combating established tumors in vivo.

### Example 3

### Materials and Methods

### Cell Culture and Description of Cell Lines

CT26 cells, 4T1 cells, and MC-38 cells were maintained in DMEM (Life Technologies) containing 10% (v/v) FCS, 1% (v/v) penicillin (10,000 IU)/streptomycin (10 mg) (BioConcept), 4.5 g/l d-glucose and 2 mM I-glutamine. cGAS KO cells were generated according to the CRISPR-Cas9 technology as described in (Ran et al., 2013). A single-guide RNA sequence targeting cGAS was cloned into the plasmid pSpCas9 (BB)-2A-GFP (PX458) (52961, AddGene), which was transfected into cells with Lipofectamine 2000 (Life Technologies). Single cells were plated into 96-well plates, expanded to obtain clones, which were validated by western blot and in functional assays for the KO phenotype. Lentiviral vectors were produced as described in (Haag et al., 2018). In brief, HEK 293T cells were transfected with pCMVDR8.74, pMD2.G plasmids and the puromycin-selectable lentiviral vector pTRIPZ containing the open reading frame of cGAS by the calcium phosphate precipitation method. The supernatant containing lentiviral particles was harvested at 48 h and 72 h, pooled and concentrated by ultracentrifugation. MC-38 cells expressing human HER2 were generated by lentiviral transduction. All cell lines used were checked for mycoplasma contamination by PCR on a regular base and no contamination was found.

cGAMP Quantification by ELISA

Cells were plated (0.06×10⁶ cells/ml) in the presence of Dox as indicated. For extracellular cGAMP measurements, cell supernatant was harvested, spun down 18,200 g at 4° C. and used directly without dilution in ELISA assay. For intracellular cGAMP measurements, cells were harvested by trypsinization (trypsin-EDTA (0.05%), Life Technologies) and collected cell pellets were lysed in RIPA lysis buffer containing 50 mM Tris, 150 mM NaCl, 1% (w/v) sodium deoxycholate, 0.03% (v/v) SDS, 0.005% (v/v) Triton X-100, 5 mM EDTA, 2 mM sodium orthovanadate and complete Protease Inhibitor Cocktail (Roche) (pellet from one well of a six-well plate in 130 μl of RIPA) for 30 min on ice. Lysed cells were centrifuged for 5 min at 18,200 g and 4° C. Diluted samples were used for cGAMP ELISA assay (Cayman 2′-3′-cGAMP ELISA kit, 501700) according to the manufacturer's instructions. Protein concentration in the supernatant was measured using BCA Pierce Protein assay kit and was used to normalize cGAMP concentration.

### MTS Assay

Cells were seeded in triplicates into 96-well plates (0.04−0.1×10⁶ cells/ml). The next day, the medium was exchanged (100 μl) and MTS reagent (10 μl/well) was added. After 1-4 h incubation at 37° C. the absorbance at 490 nm was measured and compared to dead cells and medium only.

### Western Blot Analysis

Protein extracts were loaded into 10% or 15% SDS-polyacrylamide gels. The primary antibody was incubated in 5% BSA diluted in PBS 1× overnight at 4° C. The secondary antibodies anti-mouse or anti-rabbit horseradish peroxidase (HRP)-conjugated antibodies were incubated for 1 h at room temperature. Proteins were visualized with the enhanced chemiluminescence substrate ECL (Pierce, Thermo Scientific) and imaged using the ChemiDox XRS Bio-Rad Imager. The follow-ing antibodies were used in this study: cGAS (1:1,000, D3080; 31659, Cell Signaling), GAPDH (1:1,000; AM4300, Life Technologies), STING (1:1,000, D2P2F; 13647, Cell Signaling) donkey anti-rabbit HRP (1:5,000; 711-036-152, Jackson ImmunoResearch) and donkey anti-mouse HRP (1:5,000; 715-036-151, Jackson ImmunoResearch).

### In Vivo Tumor Growth Studies

For 4T1 cells, 5×10⁵ cells were injected in 100 μl of Matrigel: PBS 1:1 were orthotopically injected into the mammary fad pad of wild-type BALB/c mice. Once tumors reached 100 mm³ on average doxycycline (0.2 mg/ml) was administered into the drinking water (precisely, for tumor growth experiments: from day 9 to day 21; for CyTOF and flow cytometry analysis: from day 9 to day 15), to induce the expression of cGAS^{WT} and cGAS^{R241E}, respectively. Tumor growth was recorded and mice were euthanized when the tumor size reached max. 1 cm³. For CT26 cells, 0.2×10⁶ cells were injected in 200 ul of Matrigel: PBS 1:1 were subcutaneously injected into wild-type BALB/c mice. In some experiments, once tumors were palpable, doxycycline (0.2 mg/ml) was administered into the drinking water from day 11 to day 21 after transplantation of tumor cells to induce the expression of cGAS^{WT} and cGAS^{R241E}, respectively.

### Analysis of Immune Cells by CyTOF and Flow Cytometry Analysis

Tumor, spleen and lymph nodes were collected for analysis. Tumor tissues were minced and digested enzymatically into single cell suspension (1 h shaking at 37°). Spleen and lymph nodes were passed through 70 μm filter to obtain a single cell suspension. Cells were counted and viability measured by PI staining. One million viable cells were aliquoted into V-bottom plated for staining. For flow cytometry staining, cells were stained for live/dead in PBS 30 min at 4° C. followed by F_{c} receptor block for 10 min on ice and incubation with surface markers antibody cocktail for 30 min at 4° C. Cells were fixed with 4% paraformaldehyde (PFA) and washed before flow cytometry acquisition using a 5 laser BD LSRFortessa. Data were analyzed using FlowJo. Cells were similarly prepared for CyTOF analysis, which was performed at the EPFL flow cytometry facility (FCCF) according to standard procedures.

Generation of cGAS Functionalized CAR T Cells

Murine T cells were isolated from Sting1 KO C57BL/6 mice and stimulated with plate-bound CD3/CD28 for 24 h. T cells were transduced twice (day 2 and day 3) with retroviruses encoding for the distinct cGAS variants as well as a human-specific HER2-CAR. T cells were culture with IL-2 (10-20 ng/ml). On day 5 transduction efficacy was quantified by flow cytometry and T cells were prepared for injection into mice.

In Vivo Testing of cGAS Functionalized CAR T Cells

Female C57BL/6 mice were injected subcutaneously with 10\hat{ }6 MC-38 colocarcinoma cells expressing HER2. Five days after tumor transplantation, mice received 2×10\hat{ }6 CAR T cells by tail vein injection or were left untreated. Some mice were treated additionally with a blocking antibody against PD-1 (100 μg) on day 1, 4, 7, and 10 using intraperitoneal injection. Tumor dimensions were measure using calipers and calculated using the formula L×W²×0.5, where L is the longest dimension and W is the perpendicular dimension.

### REFERENCES

Haag, S.M., Gulen, M.F., Reymond, L., Gibelin, A., Abrami, L., Decout, A., Heymann, M., Goot, F.G.V., Turcatti, G., Behrendt, R., and Ablasser, A. (2018). Targeting STING with covalent small-molecule inhibitors. Nature 559, 269-273. 10.1038/s41586-018-0287-8.
Ran, F.A., Hsu, P.D., Wright, J., Agarwala, V., Scott, D.A., and Zhang, F. (2013). Genome engineering using the CRISPR-Cas9 system. Nat Protoc 8, 2281-2308. 10.1038/nprot.2013.143.
Xia, T., Konno, H., Ahn, J., and Barber, G.N. (2016). Deregulation of STING Signaling in Colorectal Carcinoma Constrains DNA Damage Responses and Correlates With Tumorigenesis. Cell Rep 14, 282-297. 10.1016/j.celrep.2015.12.029.

1. Anti-tumor lymphocytes, and
a variant cyclic GMP-AMP synthase (cGAS) carrying an amino acid substitution of one or both arginines at amino acid positions 255 and 236 and/or an amino acid substitution of one or both lysines at amino acid positions 254 and 258; or a nucleic acid molecule encoding said variant cGAS
for use in the treatment of a tumor in a subject.

2. Anti-tumor lymphocytes and variant cGAS or nucleic acid molecule encoding said variant cGAS for use of claim 1, wherein the lymphocytes are T-cells or NK cells.

3. Anti-tumor lymphocytes and variant cGAS or nucleic acid molecule encoding said variant cGAS for use of claim 1, wherein the lymphocytes are chimeric antigen receptor T-cells (CAR T-cells), T-cell-receptor-engineered T-cells (TCR T-cells), chimeric antigen receptor NK-cells (CAR NK-cells), NK cell receptor-engineered NK cells (NCR NK-cells), TCR/CAR hybrid T-cells, NCR/CAR hybrid NK-cells or tumor-infiltrating lymphocytes (TILs).

4. Anti-tumor lymphocytes and variant cGAS or nucleic acid molecule encoding said variant cGAS for use of claim 3, wherein the tumor-infiltrating lymphocytes are tumor-infiltrating T-cells or tumor-infiltrating NK cells.

5. Anti-tumor lymphocytes and variant cGAS or nucleic acid molecule encoding said variant cGAS for use of any one of claims 1 to 4, wherein the anti-tumor lymphocytes are autologous anti-tumor lymphocytes.

6. Anti-tumor lymphocytes and variant cGAS or nucleic acid molecule encoding said variant cGAS for use of any one of claims 1 to 5, wherein the arginine at amino acid position 255 is substituted.

7. Anti-tumor lymphocytes and variant cGAS or nucleic acid molecule encoding said variant cGAS for use of any one of claims 1 to 6, wherein one or both arginines at amino acid positions 255 and 236 and/or the one or both lysines at amino acid positions 254 and 258 is/are substituted by a non-conservative amino acid, preferably by an amino acid with a hydrophobic side chain and most preferably by alanine or glutamic acid.

8. Anti-tumor lymphocytes and variant cGAS or nucleic acid molecule encoding said variant cGAS for use of any one of claims 1 to 7, wherein the tumor is cancer, preferably a solid cancer.

9. Anti-tumor lymphocytes and variant cGAS or nucleic acid molecule encoding said variant cGAS for use of any one of claims 1 to 8, wherein the subject is human.

10. Anti-tumor lymphocytes and variant cGAS or nucleic acid molecule encoding said variant cGAS for use of any one of claims 1 to 9,
wherein the variant cyclic GMP-AMP synthase (cGAS) carrying an amino acid substitution of one or both arginines at amino acid positions 255 or 236 and/or the one or both lysines at amino acid positions 254 and 258 comprises of consists of
(a) an amino acid sequence selected from any one of SEQ ID NOs 1 to 3 and SEQ ID NOs 12 to 23, or
(b) an amino acid sequence sharing at least 80%, preferably at least 90% identity with SEQ ID NO: 4, provided that one or both arginines at amino acid positions 255 or 236 and/or one or both lysines at amino acid positions 254 and 258 are substituted by another amino acid;
(c) the amino acid sequence of (a) or (b) further comprising a nuclear localization sequence (NLS), or
(d) the amino acid sequence of any one of (a) to (c), wherein a part of or the complete IDR is deleted; and/or
wherein the nucleic acid molecule encoding said variant cGAS comprises of consists of
(c) a nucleotide sequence selected from any one of SEQ ID NOs 5 to 7 and SEQ ID NOs 24 to 35, or
(d) a nucleotide sequence sharing at least 80%, preferably at least 90% identity with SEQ ID NO: 8, provided that the base triplet(s) encoding one or both arginines at amino acid positions 255 or 236 and/or the one or both lysines at amino acid positions 254 and 258 are substituted by another amino acid;
(c) the nucleotide sequence or (a) or (b) further encoding a nuclear localization sequence (NLS), or
(d) the nucleotide sequence of any one of (a) to (c), wherein a part of or the complete nucleotide sequence encoding the IDR is deleted.

11. Anti-tumor lymphocytes and variant cGAS or nucleic acid molecule encoding said variant cGAS for use of any one of claims 1 to 10, wherein the nucleic acid molecule encoding said variant cGAS is RNA or DNA.

12. Anti-tumor lymphocytes and variant cGAS or nucleic acid molecule encoding said variant cGAS for use of any one of claims 1 to 11, wherein the nucleic acid molecule encoding said variant cGAS is comprised in lipid nonoparticles (LNP), wherein the LNPs were preferably contacted with the anti-tumor lymphocytes prior to their use in the treatment of a tumor in a subject, so that the nucleic acid molecule encoding said variant cGAS and the LNPs were released into the cytoplasm of the anti-tumor lymphocytes.

13. Anti-tumor lymphocytes and variant cGAS or nucleic acid molecule encoding said variant cGAS for use of any one of claims 1 to 11, wherein the nucleic acid molecule encoding said variant cGAS is an expression vector, preferably an expression vector within the anti-tumor lymphocytes.

14. Anti-tumor lymphocytes and variant cGAS or nucleic acid molecule encoding said variant cGAS for use of any one of claims 1 to 11, wherein the nucleic acid molecule encoding said variant cGAS is within the genome of the anti-tumor lymphocytes.

15. Anti-tumor lymphocytes and variant cGAS or nucleic acid molecule encoding said variant cGAS for use of claim 14, wherein the nucleic acid molecule encoding said variant cGAS has been inserted into the genome of the anti-tumor lymphocytes by a genome editing technology, such as meganuclease, Zn-finger, TALEN or CRISPR.


SEQUENCE LISTING (35 sequences)

SEQ ID NO: 1 - protein, 522 aa, synthetic construct
  REGION 1..522: note = R255A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKANPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 2 - protein, 522 aa, synthetic construct
  REGION 1..522: note = R236A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 3 - protein, 522 aa, synthetic construct
  REGION 1..522: note = R236A/R255A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFKANPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 4 - protein, 522 aa, Homo sapiens
  REGION 1..522: note = WT cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 5 - DNA, 1569 bp, synthetic construct
  misc_feature 1..1569: note = R255A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaagcaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 6 - DNA, 1569 bp, synthetic construct
  misc_feature 1..1569: note = R236A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 7 - DNA, 1569 bp, synthetic construct
  misc_feature 1..1569: note = R236A/R255A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttaaagcaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 8 - DNA, 1569 bp, Homo sapiens
  misc_feature 1..1569: note = WT cGAS nt sequence
  source 1..1569: mol_type = genomic DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 9 - protein, 7 aa, synthetic construct
  REGION 1..7: note = monopartite nuclear localization sequence
PKKKRKV

SEQ ID NO: 10 - DNA, 21 bp, synthetic construct
  misc_feature 1..21: note = nucleotide sequence of the monopartite nuclear localization sequence
  source 1..21: mol_type = other DNA
cctaagaagaagaggaaggtt

SEQ ID NO: 11 - protein, 16 aa, synthetic construct
  REGION 1..16: note = bipartite nuclear localization sequence
KRPAATKKAGQAKKKK

SEQ ID NO: 12 - protein, 522 aa, synthetic construct
  REGION 1..522: note = K254A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFARNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 13 - protein, 522 aa, synthetic construct
  REGION 1..522: note = K258A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPAENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 14 - protein, 522 aa, synthetic construct
  REGION 1..522: note = R236A/K254A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFARNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 15 - protein, 522 aa, synthetic construct
  REGION 1..522: note = R236A/K258A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFKRNPAENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 16 - protein, 522 aa, synthetic construct
  REGION 1..522: note = K254A/R255A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFAANPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 17 - protein, 522 aa, synthetic construct
  REGION 1..522: note = K254A/K258A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFARNPAENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 18 - protein, 522 aa, synthetic construct; identical to SEQ ID NO: 17
  REGION 1..522: note = R255A/K258A cGAS AA sequence

SEQ ID NO: 19 - protein, 522 aa, synthetic construct
  REGION 1..522: note = R236A/K254A/R255A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFAANPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 20 - protein, 522 aa, synthetic construct
  REGION 1..522: note = R236A/K254A/K258A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFARNPAENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 21 - protein, 522 aa, synthetic construct
  REGION 1..522: note = R236A/R255A/K258A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFKANPAENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 22 - protein, 522 aa, synthetic construct
  REGION 1..522: note = K254A/R255A/K258A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFAANPAENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 23 - protein, 522 aa, synthetic construct
  REGION 1..522: note = R236A/K254A/R255A/K258A cGAS AA sequence
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPAIQLEEYSNTRAYYFVKFAANPAENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 24 - DNA, 1569 bp, synthetic construct
  misc_feature 1..1569: note = K254A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttgcaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 25 - DNA, 1569 bp, synthetic construct
  misc_feature 1..1569: note = K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaaagaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 26 - DNA, 1569 bp, synthetic construct
  misc_feature 1..1569: note = R236A/K254A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttgcaagaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 27 - DNA, 1569 bp, synthetic construct
  misc_feature 1..1569: note = R236A/K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttaaaagaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 28 - DNA, 1569 bp, synthetic construct
  misc_feature 1..1569: note = K254A/R255A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttgcagcaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 29 - DNA, 1569 bp, synthetic construct
  misc_feature 1..1569: note = K254A/K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttgcaagaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 30 - DNA, 1569 bp, synthetic construct
  misc_feature 1..1569: note = R255A/K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttaaagcaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 31 - DNA, 1569 bp, synthetic construct
  misc_feature 1..1569: note = R236A/K254A/R255A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttgcagcaaatccgaaagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 32 - DNA, 1569 bp, synthetic construct
  misc_feature 1..1569: note = R236A/K254A/K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttgcaagaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 33 - DNA, 1569 bp, synthetic construct
  misc_feature 1..1569: note = R236A/R255A/K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttaaagcaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 34 - DNA, 1569 bp, synthetic construct
  misc_feature 1..1569: note = K254A/R255A/K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccagaattcaactagaagaatattccaacactcgtgcatattactttgtgaaatttgcagcaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga

SEQ ID NO: 35 - DNA, 1569 bp, synthetic construct
  misc_feature 1..1569: note = R236A/K254A/R255A/K258A cGAS nt sequence
  source 1..1569: mol_type = other DNA
atgcagccttggcacggaaaggccatgcagagagcttccgaggccggagccactgcccccaaggcttccgcacggaatgccaggggcgccccgatggatcccaccgagtctccggctgcccccgaggccgccctgcctaaggcgggaaagttcggccccgccaggaagtcgggatcccggcagaaaaagagcgccccggacacccaggagaggccgcccgtccgcgcaactggggcccgcgccaaaaaggcccctcagcgcgcccaggacacgcagccgtctgacgccaccagcgcccctggggcagaggggctggagcctcctgcggctcgggagccggctctttccagggctggttcttgccgccagaggggcgcgcgctgctccacgaagccaagacctccgcccgggccctgggacgtgcccagccccggcctgccggtctcggcccccattctcgtacggagggatgcggcgcctggggcctcgaagctccgggcggttttggagaagttgaagctcagccgcgatgatatctccacggcggcggggatggtgaaaggggttgtggaccacctgctgctcagactgaagtgcgactccgcgttcagaggcgtcgggctgctgaacaccgggagctactatgagcacgtgaagatttctgcacctaatgaatttgatgtcatgtttaaactggaagtccccgcaattcaactagaagaatattccaacactcgtgcatattactttgtgaaatttgcagcaaatccggcagaaaatcctctgagtcagtttttagaaggtgaaatattatcagcttctaagatgctgtcaaagtttaggaaaatcattaaggaagaaattaacgacattaaagatacagatgtcatcatgaagaggaaaagaggagggagccctgctgtaacacttcttattagtgaaaaaatatctgtggatataaccctggctttggaatcaaaaagtagctggcctgctagcacccaagaaggcctgcgcattcaaaactggctttcagcaaaagttaggaagcaactacgactaaagccattttaccttgtacccaagcatgcaaaggaaggaaatggtttccaagaagaaacatggcggctatccttctctcacatcgaaaaggaaattttgaacaatcatggaaaatctaaaacgtgctgtgaaaacaaagaagagaaatgttgcaggaaagattgtttaaaactaatgaaataccttttagaacagctgaaagaaaggtttaaagacaaaaaacatctggataaattctcttcttatcatgtgaaaactgccttctttcacgtatgtacccagaaccctcaagacagtcagtgggaccgcaaagacctgggcctctgctttgataactgcgtgacatactttcttcagtgcctcaggacagaaaaacttgagaattattttattcctgaattcaatctattctctagcaacttaattgacaaaagaagtaaggaatttctgacaaagcaaattgaatatgaaagaaacaatgagtttccagtttttgatgaattttga